(19) United States
(12) Patent Application Publication
(10) Pub. No.: US 2025/0075186 A1
(43) Pub. Date: Mar. 6, 2025

(54) In Vitro and In Vivo Generation of Insulin Producing Cells

(71) Applicant: CREATIVE MEDICAL TECHNOLOGIES, INC., Phoenix, AZ (US)

(72) Inventors: Thomas Ichim, San Diego, CA (US); Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/785,132

(22) Filed: Jul. 26, 2024

Related U.S. Application Data
(60) Provisional application No. 63/580,657, filed on Sep. 5, 2023.

(51) Int. Cl. C12N 5/071; C07K 14/62; C12N 15/86

(52) U.S. Cl. CPC C12N 5/0676; C07K 14/62; C12N 15/86; C12N 2501/603; C12N 2506/45

(57) ABSTRACT

Methods and compositions of matter for production of pancreatic beta cells, as well as beta-like cells that are capable of producing insulin. Pluripotent stem cells are generated through either viral vector or non-viral vector means and induced into differentiation pathways through environments resembling the pluripotent pancreatic environment. In vivo transfection of dedifferentiation and beta cell differentiation genes are provided in vivo in the pancreatic microenvironment to generate de novo beta cells and islets from differentiated tissue or islet progenitors. Means of inducing differentiation of islet progenitors in vivo are also provided.

### CROSS-REFERENCE TO RELATED APPLICATIONS

The present application claims benefit of U.S. Provisional Patent Application Ser. No. 63/580,657, filed on Aug. 5, 2023, titled IN VITRO AND IN VIVO GENERATION OF INSULIN PRODUCING CELLS, the contents of which are incorporated herein by reference in its entirety.

### FIELD OF THE INVENTION

The invention pertains to the field of diabetes, more particularly the invention pertains to the field of treatment of diabetes through replacement of insulin producing cells, more particularly the invention provides means of generation of insulin producing cells in vitro or in vivo.

### BACKGROUND OF THE INVENTION

The shortage of cadaveric human islets necessitates an alternative source for islets. Islets derived from differentiation of human pluripotent stem cells are promising islet cell sources, however, there are numerous limitations that prevent clinical translation, specifically, inefficiency of generating pluripotent stem cells, difficulty in prevention of ectopic tissue formation, and issues of immunological rejection as a result of autoimmunity and alloimmunity.

A tissue microenvironment plays a paramount role in in vitro islet generation. Tissue niches such as insoluble signals, including extracellular matrix (ECM), coordinate with soluble signals such as growth factors, to regulate cell proliferation, differentiation, and maturation. To data recapitulation of tissue microenvironment has not been utilized in generation of pluripotent derived insulin producing cells.

The generation of insulin producing cells in vitro allows for creating cells that can be administered to the diabetic patient, however, the only commonly used methods, intrapancreatic and intrahepatic are limited by poor engraftment, accordingly, the current invention teaches means of in vivo generation of pluripotent stem cells and differentiation into beta cells or “beta-like” cells which are capable of producing insulin in a regulated manner.

### SUMMARY OF THE INVENTION

Preferred embodiments include methods of generating a population of insulin producing cells, said method comprising of: a) obtaining a somatic cell; b) dedifferentiating said somatic cell into a pluripotent stem cell; b) differentiating said pluripotent stem cell into a mesendoderm cell; c) differentiating said mesendoderm cell into an endoderm cell; and d) differentiating said endoderm cell into a beta cell, or beta-like cell.

Preferred methods include embodiments wherein said pluripotent stem cells express one or more genes selected from a group comprising of: a) SSEA4; b) OCT4; c) NANOG; and d) PIM-1.

Preferred methods include embodiments wherein said pluripotent stem cells express KLF4.

Preferred methods include embodiments wherein said pluripotent stem cells express Sox-2.

Preferred methods include embodiments wherein said pluripotent stem cells express k-RAS.

Preferred methods include embodiments wherein said pluripotent stem cells express son of sevenless.

Preferred methods include embodiments wherein said mesendoderm cell expresses MIXL1.

Preferred methods include embodiments wherein said mesendoderm cell expresses CD15.

Preferred methods include embodiments wherein said mesendoderm cell expresses c-met.

Preferred methods include embodiments wherein said mesendoderm cell expresses c-kit.

Preferred methods include embodiments wherein said mesendoderm cell expresses brachyury.

Preferred methods include embodiments wherein said mesendoderm cell expresses IL-3 receptor.

Preferred methods include embodiments wherein said endoderm cell expresses IL-6 receptor.

Preferred methods include embodiments wherein said endoderm cell expresses Goosecoid.

Preferred methods include embodiments wherein said endoderm cell expresses PDX-1.

Preferred methods include embodiments wherein said endoderm cell expresses FoxA2.

Preferred methods include embodiments wherein said endoderm cell expresses CXCR4.

Preferred methods include embodiments wherein said endoderm cell expresses Sox17.

Preferred methods include embodiments wherein said endoderm cell expresses Hnf6.

Preferred methods include embodiments wherein said endoderm cell expresses Ngn3.

Preferred methods include embodiments wherein said endoderm cell expresses NeuroD1.

Preferred methods include embodiments wherein said endoderm cell expresses MafB.

Preferred methods include embodiments wherein said endoderm cell expresses Nkx2.2.

Preferred methods include embodiments wherein said endoderm cell expresses MafA1.

Preferred methods include embodiments wherein said endoderm cell expresses Nkx2.1

Preferred methods include embodiments wherein said endoderm cell expresses PANC1.

Preferred methods include embodiments wherein said pluripotent stem cell is contacted with one or more factors capable of inducing differentiation.

Preferred methods include embodiments wherein said pluripotent stem cell is induced to differentiate by culture with WNT3A.

Preferred methods include embodiments wherein said pluripotent stem cell is induced to differentiate by culture with WNT3A and interleukin-7.

Preferred methods include embodiments wherein said pluripotent stem cell is induced to differentiate by culture with WNT3A and interleukin-7 and hepatocyte growth factor.

Preferred methods include embodiments wherein said pluripotent stem cell is induced to differentiate by culture with WNT3A and hepatocyte growth factor.

Preferred methods include embodiments wherein said pluripotent stem cell is induced to differentiate by culture with WNT3A and hepatocyte growth factor and interleukin-7 in the presence of endothelial cells.

Preferred methods include embodiments wherein said endothelial cells are generated from bone marrow sources.

Preferred methods include embodiments wherein said endothelial cells are generated by extraction of CD133 expressing cells from said bone marrow mononuclear cells.

Preferred methods include embodiments wherein said extraction of CD133 expressing cells is performed from mononuclear cells that are plastic adherent.

Preferred methods include embodiments wherein said extraction of CD133 expressing cells is performed from mononuclear cells that express NGF receptor.

Preferred methods include embodiments wherein said extraction of CD133 expressing cells is performed from mononuclear cells that express c-kit.

Preferred methods include embodiments wherein said extraction of CD133 expressing cells is performed from mononuclear cells that express CD14.

Preferred methods include embodiments wherein said extraction of CD133 expressing cells is performed from mononuclear cells that express EGF receptor.

Preferred methods include embodiments wherein said extraction of CD133 expressing cells is performed from mononuclear cells that express c-met.

Preferred methods include embodiments wherein said endothelial cells are generated by extraction of CD34 expressing cells from said bone marrow mononuclear cells.

Preferred methods include embodiments wherein said extraction of CD34 expressing cells is performed from mononuclear cells that are plastic adherent.

Preferred methods include embodiments wherein said extraction of CD34 expressing cells is performed from mononuclear cells that express NGF receptor.

Preferred methods include embodiments wherein said extraction of CD34 expressing cells is performed from mononuclear cells that express c-kit.

Preferred methods include embodiments wherein said extraction of CD34 expressing cells is performed from mononuclear cells that express CD14.

Preferred methods include embodiments wherein said extraction of CD34 expressing cells is performed from mononuclear cells that express EGF receptor.

Preferred methods include embodiments wherein said extraction of CD34 expressing cells is performed from mononuclear cells that express c-met.

Preferred methods include embodiments wherein said endothelial cells are generated by extraction of EPCAM expressing cells from said bone marrow mononuclear cells.

Preferred methods include embodiments wherein said extraction of EPCAM expressing cells is performed from mononuclear cells that are plastic adherent.

Preferred methods include embodiments wherein said extraction of EPCAM expressing cells is performed from mononuclear cells that express NGF receptor.

Preferred methods include embodiments wherein said extraction of EPCAM expressing cells is performed from mononuclear cells that express c-kit.

Preferred methods include embodiments wherein said extraction of EPCAM expressing cells is performed from mononuclear cells that express CD14.

Preferred methods include embodiments wherein said extraction of EPCAM expressing cells is performed from mononuclear cells that express EGF receptor.

Preferred methods include embodiments wherein said extraction of EPCAM expressing cells is performed from mononuclear cells that express c-met.

Preferred methods include embodiments wherein said endothelial cells are derived from placental tissue.

Preferred methods include embodiments wherein said endothelial cells are derived from umbilical cord tissue.

Preferred methods include embodiments wherein said endothelial cells are derived from Wharton's Jelly.

Preferred methods include embodiments wherein said endothelial cells are derived from endometrial tissue.

Preferred methods include embodiments wherein said endothelial cells are derived from omental tissue.

Preferred methods include embodiments wherein said endothelial cells are derived from umbilical cord blood CD34 cells.

Preferred methods include embodiments wherein said endothelial cells are derived from umbilical cord blood CD33 cells.

Preferred methods include embodiments wherein said endothelial cells are derived from umbilical cord blood CD133 cells.

Preferred methods include embodiments wherein said endothelial cells are derived from umbilical cord blood CD73 cells.

Preferred methods include embodiments wherein said endothelial cells are derived from umbilical cord blood EGF receptor expressing cells.

Preferred methods include embodiments wherein said endothelial cells are derived from umbilical cord blood GM-CSF stimulated mononuclear cells.

Preferred methods include embodiments wherein said mononuclear cells are purified for expression of CD133.

Preferred methods include embodiments wherein said mononuclear cells are purified for expression of CD133 and CD73.

Preferred methods include embodiments wherein said mononuclear cells are purified for expression of CD133 and lacking CD45.

Preferred methods include embodiments wherein said mononuclear cells are purified for expression of CD133 and lacking HLA-DR.

Preferred methods include embodiments wherein said endothelial cells are engineered to express molecules that induce differentiation of pluripotent stem cells into endoderm lineage.

Preferred methods include embodiments wherein said endothelial cells are engineered to express one or more members of the transforming growth factor beta superfamily of cytokines.

Preferred methods include embodiments wherein said member of the transforming growth factor beta superfamily of cytokines is activin A.

Preferred methods include embodiments wherein said member of the transforming growth factor beta superfamily of cytokines is activin AB.

Preferred methods include embodiments wherein said member of the transforming growth factor beta superfamily of cytokines is activin AC.

Preferred methods include embodiments wherein said member of the transforming growth factor beta superfamily of cytokines is activin B.

Preferred methods include embodiments wherein said member of the transforming growth factor beta superfamily of cytokines is activin C.

Preferred methods include embodiments wherein said member of the transforming growth factor beta superfamily of cytokines is C17orf99.

Preferred methods include embodiments wherein said member of the transforming growth factor beta superfamily of cytokines is INHBA.

Preferred methods include embodiments wherein said member of the transforming growth factor beta superfamily of cytokines is INHBB.

Preferred methods include embodiments wherein said member of the transforming growth factor beta superfamily of cytokines is INHBE.

Preferred methods include embodiments wherein said member of the transforming growth factor beta superfamily of cytokines is inhibin.

Preferred methods include embodiments wherein said member of the transforming growth factor beta superfamily of cytokines is inhibin A.

Preferred methods include embodiments wherein said member of the transforming growth factor beta superfamily of cytokines is inhibin B.

Preferred methods include embodiments wherein said member of the transforming growth factor beta superfamily of cytokines is PCP/BMP1.

Preferred methods include embodiments wherein said member of the transforming growth factor beta superfamily of cytokines is BMP2

Preferred methods include embodiments wherein said member of the transforming growth factor beta superfamily of cytokines is BMP2/BMP4 heterodimer.

Preferred methods include embodiments wherein said member of the transforming growth factor beta superfamily of cytokines is BMP2/BMP6 heterodimer.

Preferred methods include embodiments wherein said member of the transforming growth factor beta superfamily of cytokines is BMP2/BMP7 heterodimer.

Preferred methods include embodiments wherein said member of the transforming growth factor beta superfamily of cytokines is BMP2a.

Preferred methods include embodiments wherein said member of the transforming growth factor beta superfamily of cytokines is BMP3.

Preferred methods include embodiments wherein said member of the transforming growth factor beta superfamily of cytokines is BMP3b/GDF-10.

Preferred methods include embodiments wherein said member of the transforming growth factor beta superfamily of cytokines is BMP4.

Preferred methods include embodiments wherein said member of the transforming growth factor beta superfamily of cytokines is BMP4/BMP7 heterodimer.

Preferred methods include embodiments wherein said member of the transforming growth factor beta superfamily of cytokines is BMP5.

Preferred methods include embodiments wherein said member of the transforming growth factor beta superfamily of cytokines is BMP6.

Preferred methods include embodiments wherein said member of the transforming growth factor beta superfamily of cytokines is BMP7.

Preferred methods include embodiments wherein said member of the transforming growth factor beta superfamily of cytokines is BMP8.

Preferred methods include embodiments wherein said member of the transforming growth factor beta superfamily of cytokines is BMP8a.

97. Preferred methods include embodiments wherein said member of the transforming growth factor beta superfamily of cytokines is BMP8b.

Preferred methods include embodiments wherein said member of the transforming growth factor beta superfamily of cytokines is BMP9.

Preferred methods include embodiments wherein said member of the transforming growth factor beta superfamily of cytokines is BMP10.

Preferred methods include embodiments wherein said member of the transforming growth factor beta superfamily of cytokines is BMP15/GDF-9B.

Preferred methods include embodiments wherein said member of the transforming growth factor beta superfamily of cytokines is decapentaplegic.

Preferred methods include embodiments wherein said member of the transforming growth factor beta superfamily of cytokines is artemin.

Preferred methods include embodiments wherein said member of the transforming growth factor beta superfamily of cytokines is GDNF.

Preferred methods include embodiments wherein said member of the transforming growth factor beta superfamily of cytokines is neurturin.

Preferred methods include embodiments wherein said member of the transforming growth factor beta superfamily of cytokines is persephin.

Preferred methods include embodiments wherein said differentiation of said pluripotent stem cell into said insulin producing cell is performed in vivo.

Preferred methods include embodiments wherein pluripotent stem cells are generated in vivo by transfection with one or more dedifferentiation factors.

Preferred methods include embodiments wherein said in vivo transfection is performed by use of ultrasound mediated transfection.

Preferred methods include embodiments wherein said in vivo transfection is performed by use of extracorporeal pulse wave mediated transfection.

Preferred methods include embodiments wherein said in vivo transfection is performed by use of electroporation mediated transfection.

Preferred methods include embodiments wherein said in vivo transfection is performed by use of hydrodynamic mediated transfection.

Preferred methods include embodiments wherein said in vivo transfection is performed by use of liposomal mediated transfection.

Preferred methods include embodiments wherein said in vivo transfection is performed by use of immunoliposomal mediated transfection.

Preferred methods include embodiments wherein said in vivo transfection is performed by use of nanoparticle mediated transfection.

The method of claim 111, wherein said nanoparticles used for transfection are cowpea mosaic virus particles.

The method of claim 103, wherein said in vivo generation of pluripotent stem cells is generated by means of local administration of exosomes from pluripotent stem cells.

Preferred methods include embodiments wherein said exosomes are approximately 50-200 nanometers in size.

Preferred methods include embodiments wherein said exosomes are approximately 70-150 nanometers in size.

Preferred methods include embodiments wherein said exosomes are approximately 80-100 nanometers in size.

Preferred methods include embodiments wherein said exosomes express calreticulin.

Preferred methods include embodiments wherein said exosomes express Annexin-V.

Preferred methods include embodiments wherein said exosomes express CD8.

Preferred methods include embodiments wherein said exosomes express CD6.

Preferred methods include embodiments wherein said exosomes express CD73.

Preferred methods include embodiments wherein said exosomes express TGF-beta.

Preferred methods include embodiments wherein said exosomes express ALIX.

Preferred methods include embodiments wherein said exosomes express aldolase A.

Preferred methods include embodiments wherein said exosomes express hsp70.

Preferred methods include embodiments wherein said exosomes express CD44.

Preferred methods include embodiments wherein said exosomes express CD63.

Preferred methods include embodiments wherein said exosomes express CD9

Preferred methods include embodiments wherein said exosomes express TSG101.

Preferred methods include embodiments wherein said exosomes express CD81.

Preferred methods include embodiments wherein said exosomes express anexa 2.

Preferred methods include embodiments wherein said dedifferentiation factor is OCT4.

Preferred methods include embodiments wherein said dedifferentiation factor is NANOG.

Preferred methods include embodiments wherein said dedifferentiation factor is LIN28.

Preferred methods include embodiments wherein said dedifferentiation factor is KLF4.

Preferred methods include embodiments wherein said dedifferentiation factor is Sox2.

Preferred methods include embodiments wherein said dedifferentiation factor is k-RAS.

Preferred methods include embodiments wherein said dedifferentiation factor is c-myc.

Preferred methods include embodiments wherein said dedifferentiation factor is OCT4 and k-RAS in the presence of endothelial cells.

Preferred methods include embodiments wherein said dedifferentiation factor is OCT4 and k-RAS in the presence of endothelial cell exosomes.

Preferred methods include embodiments wherein said dedifferentiation factor is OCT4 and k-RAS in the presence of fibroblast cells.

Preferred methods include embodiments wherein said fibroblast cells are transfected with LIF.

Preferred methods include embodiments wherein said fibroblast cells are transfected with NANOG.

Preferred methods include embodiments wherein said fibroblast cells are transfected with hepatocyte growth factor.

Preferred methods include embodiments wherein said fibroblast cells are transfected with activin A.

Preferred methods include embodiments wherein said fibroblast cells are transfected with endoglin.

Preferred methods include embodiments wherein said fibroblast cells are transfected with angiopoietin.

Preferred methods include embodiments wherein said dedifferentiation factor is OCT4 and k-RAS in the presence of mesenchymal stem cells.

The method of claim 148, wherein said mesenchymal stem cells are derived from a source selected from a group of sources comprising of: a) bone marrow; b) umbilical cord blood; c) peripheral cord blood; d) menstrual blood; e) Wharton's Jelly; f) Perinatal Tissue.

The method of claim 149, wherein said mesenchymal stem cells are selected based on expression of CD73.

Preferred methods include embodiments wherein said CD73 cells are expanded in the presence of PDGF.

Preferred methods include embodiments wherein said CD73 cells are expanded in the presence of platelet lysate.

Preferred methods include embodiments wherein said CD73 cells are expanded in the presence of EGF.

Preferred methods include embodiments wherein said CD73 cells are expanded in the presence of FGF-1.

Preferred methods include embodiments wherein said CD73 cells are expanded in the presence of FGF-2.

Preferred methods include embodiments wherein said CD73 cells are expanded in the presence of FGF-5.

Preferred methods include embodiments wherein said CD73 cells are expanded in the presence of VEGF.

Preferred methods include embodiments wherein said CD73 cells are expanded in the presence of VEGF-C.

Preferred methods include embodiments wherein said CD73 cells are expanded in the presence of CTNF.

Preferred methods include embodiments wherein said CD73 cells are expanded in the presence of PDGF and a histone deacetylase inhibitor.

Preferred methods include embodiments wherein said CD73 cells are expanded in the presence of EGF and a histone deacetylase inhibitor.

Preferred methods include embodiments wherein said CD73 cells are expanded in the presence of FGF-1 and a histone deacetylase inhibitor.

Preferred methods include embodiments wherein said CD73 cells are expanded in the presence of FGF-2 and a histone deacetylase inhibitor.

Preferred methods include embodiments wherein said CD73 cells are expanded in the presence of BDNF and a histone deacetylase inhibitor.

Preferred methods include embodiments wherein said histone deacetylase inhibitor is valproic acid.

Preferred methods include embodiments wherein said valproic acid is added at a concentration and time period sufficient to induce production of IL-10 from said mesenchymal stem cells at a concentration of more than 10 ng per million cells.

Preferred methods include embodiments wherein said valproic acid is added at a concentration and time period sufficient to induce production of IL-10 from said mesenchymal stem cells at a concentration of more than 50 ng per million cells.

Preferred methods include embodiments wherein said valproic acid is added at a concentration and time period sufficient to induce production of IL-10 from said mesenchymal stem cells at a concentration of more than 100 ng per million cells.

Preferred methods include embodiments wherein said histone deacetylase inhibitor is phenylbutyrate.

Preferred methods include embodiments wherein said phenylbutyrate is at a concentration and time period sufficient to induce production of IL-10 from said mesenchymal stem cells at a concentration of more than 10 ng per million cells.

Preferred methods include embodiments wherein said phenylbutyrate is added at a concentration and time period sufficient to induce production of IL-10 from said mesenchymal stem cells at a concentration of more than 50 ng per million cells.

Preferred methods include embodiments wherein said phenylbutyrate is added at a concentration and time period sufficient to induce production of IL-10 from said mesenchymal stem cells at a concentration of more than 100 ng per million cells.

Preferred methods include embodiments wherein said histone deacetylase inhibitor is trichostatin A.

Preferred methods include embodiments wherein said trichostatin A is added to said cells at a concentration and time period sufficient to induce production of IL-10 from said mesenchymal stem cells at a concentration of more than 10 ng per million cells.

Preferred methods include embodiments wherein said trichostatin A is added at a concentration and time period sufficient to induce production of IL-10 from said mesenchymal stem cells at a concentration of more than 50 ng per million cells.

Preferred methods include embodiments wherein said trichostatin A is added at a concentration and time period sufficient to induce production of IL-10 from said mesenchymal stem cells at a concentration of more than 100 ng per million cells.

Preferred methods include embodiments wherein said histone deacetylase inhibitor is vorinostat.

Preferred methods include embodiments wherein said vorinostat is added to said cells at a concentration and time period sufficient to induce production of IL-10 from said mesenchymal stem cells at a concentration of more than 10 ng per million cells.

Preferred methods include embodiments wherein said vorinostat is added at a concentration and time period sufficient to induce production of IL-10 from said mesenchymal stem cells at a concentration of more than 50 ng per million cells.

Preferred methods include embodiments wherein said vorinostat is added at a concentration and time period sufficient to induce production of IL-10 from said mesenchymal stem cells at a concentration of more than 100 ng per million cells.

Preferred methods include embodiments wherein said histone deacetylase inhibitor is panobinostat.

Preferred methods include embodiments wherein said panobinostat is added to said cells at a concentration and time point sufficient to induce production of IL-10 from said mesenchymal stem cells at a concentration of more than 10 ng per million cells.

Preferred methods include embodiments wherein said panobinostat is added at a concentration and time period sufficient to induce production of IL-10 from said mesenchymal stem cells at a concentration of more than 50 ng per million cells.

Preferred methods include embodiments wherein said panobinostat is added at a concentration and time period sufficient to induce production of IL-10 from said mesenchymal stem cells at a concentration of more than 100 ng per million cells.

Preferred methods include embodiments wherein said histone deacetylase inhibitor is entinostat.

Preferred methods include embodiments wherein said entinostat is added to said cells at a concentration and time point sufficient to induce production of IL-10 from said mesenchymal stem cells at a concentration of more than 10 ng per million cells.

Preferred methods include embodiments wherein said entinostat is added at a concentration and time period sufficient to induce production of IL-10 from said mesenchymal stem cells at a concentration of more than 50 ng per million cells.

Preferred methods include embodiments wherein said entinostat is added at a concentration and time period sufficient to induce production of IL-10 from said mesenchymal stem cells at a concentration of more than 100 ng per million cells.

Preferred methods include embodiments wherein said histone deacetylase inhibitor is romidepsin.

Preferred methods include embodiments wherein said romidepsin is added to said cells at a concentration and time point sufficient to induce production of IL-10 from said mesenchymal stem cells at a concentration of more than 10 ng per million cells.

Preferred methods include embodiments wherein said romidepsin is added at a concentration and time period sufficient to induce production of IL-10 from said mesenchymal stem cells at a concentration of more than 50 ng per million cells.

Preferred methods include embodiments wherein said romidepsin is added at a concentration and time period sufficient to induce production of IL-10 from said mesenchymal stem cells at a concentration of more than 100 ng per million cells.

Preferred methods include embodiments wherein said histone deacetylase inhibitor is belinostat.

Preferred methods include embodiments wherein said belinostat is added to said cells at a concentration and time point sufficient to induce production of IL-10 from said mesenchymal stem cells at a concentration of more than 10 ng per million cells.

Preferred methods include embodiments wherein said belinostat is added at a concentration and time period sufficient to induce production of IL-10 from said mesenchymal stem cells at a concentration of more than 50 ng per million cells.

Preferred methods include embodiments wherein said belinostat is added at a concentration and time period sufficient to induce production of IL-10 from said mesenchymal stem cells at a concentration of more than 100 ng per million cells.

Preferred methods include embodiments wherein said histone deacetylase inhibitor is tubastatin.

Preferred methods include embodiments wherein said tubastatin is added to said cells at a concentration and time point sufficient to induce production of IL-10 from said mesenchymal stem cells at a concentration of more than 10 ng per million cells.

Preferred methods include embodiments wherein said tubastatin is added at a concentration and time period sufficient to induce production of IL-10 from said mesenchymal stem cells at a concentration of more than 50 ng per million cells.

Preferred methods include embodiments wherein said tubastatin is added at a concentration and time period sufficient to induce production of IL-10 from said mesenchymal stem cells at a concentration of more than 100 ng per million cells.

Preferred methods include embodiments wherein said histone deacetylase inhibitor is decitabine.

Preferred methods include embodiments wherein said decitabine is added to said cells at a concentration and time point sufficient to induce production of IL-10 from said mesenchymal stem cells at a concentration of more than 10 ng per million cells.

Preferred methods include embodiments wherein said decitabine is added at a concentration and time period sufficient to induce production of IL-10 from said mesenchymal stem cells at a concentration of more than 50 ng per million cells.

Preferred methods include embodiments wherein said decitabine is added at a concentration and time period sufficient to induce production of IL-10 from said mesenchymal stem cells at a concentration of more than 100 ng per million cells.

Preferred methods include embodiments wherein said insulin producing cells are generated in a manner so as to reduce immunogenicity.

Preferred methods include embodiments wherein said insulin producing cells are generated in a manner so as to enhance tolerogenicity.

Preferred methods include embodiments wherein said insulin producing cells are gene edited to remove immunogenic molecules.

Preferred methods include embodiments wherein said immunogenic molecule is HLA.

Preferred methods include embodiments wherein said immunogenic molecule is CD40.

Preferred methods include embodiments wherein said immunogenic molecule is CD80.

Preferred methods include embodiments wherein said immunogenic molecule is CD86.

Preferred methods include embodiments wherein said immunogenic molecule is IL-12.

Preferred methods include embodiments wherein said immunogenic molecule is HMGB1.

Preferred methods include embodiments wherein said immunogenic molecule is TLR3.

Preferred methods include embodiments wherein said immunogenic molecule is TLR4.

Preferred methods include embodiments wherein said immunogenic molecule is TLR9.

Preferred methods include embodiments wherein said tolerogenicity is enhanced by transfection with Fas ligand.

Preferred methods include embodiments wherein said tolerogenicity is enhanced by transfection with IL-2.

Preferred methods include embodiments wherein said tolerogenicity is enhanced by transfection with AIRE.

Preferred methods include embodiments wherein said tolerogenicity is enhanced by transfection with FoxP3.

Preferred methods include embodiments wherein said tolerogenicity is enhanced by transfection with TGF-beta.

Preferred methods include embodiments wherein said tolerogenicity is enhanced by transfection with endoglin.

Preferred methods include embodiments wherein said tolerogenicity is enhanced by transfection with angiopoietin.

Preferred methods include embodiments wherein said tolerogenicity is enhanced by transfection with IL-4.

Preferred methods include embodiments wherein said tolerogenicity is enhanced by transfection with IL-10.

Preferred methods include embodiments wherein said tolerogenicity is enhanced by transfection with HLA-G.

Preferred methods include embodiments wherein said tolerogenicity is enhanced by transfection with IL-35.

Preferred methods include embodiments wherein said tolerogenicity is enhanced by transfection with TIM-3.

Preferred methods include embodiments wherein said tolerogenicity is enhanced by transfection with LAG-3.

Preferred methods include embodiments wherein said tolerogenicity is enhanced by transfection with extracellular vimentin.

Preferred methods include embodiments wherein said tolerogenicity is enhanced by transfection with calreticulin.

Preferred methods include embodiments wherein said pluripotent stem cells are created using non-integrating viral vectors.

Preferred methods include embodiments wherein said pluripotent stem cells result in no karyotype abnormality.

Preferred methods include embodiments wherein said pluripotent stem cells can be differentiated and demonstrate no adventitial viral components.

Preferred methods include embodiments wherein said pluripotent stem cells can be differentiated and produce functional insulin.

### DETAILED DESCRIPTION OF THE INVENTION

The invention provides means of generating pancreatic beta cell and “beta-like” cells in vitro and in vivo. In one embodiment the invention provides protocols a “graded” dedifferentiation and a graded “re differentiation” of cells to beta cells. In vitro and in vivo generation of beta cells is performed in a matter that resembles fetal development of pancreatic beta cells.

The term “DNA damage response” refers to any process that results in a change in state or activity of a cell (in terms of movement, secretion, enzyme production, gene expression, etc.) as a result of a stimulus, indicating damage to its DNA from environmental insults or errors during metabolism.

The term “apoptosis response” refers to a process that results in apoptosis of a cell, for example in response to DNA damage. A lower apoptotic rate or a failure of a cell to apoptose at all (collectively referred to a reduced apoptosis response) is associated with uncontrolled cell proliferation and more specifically with malignancy.

The term “polyploidy” refers to the condition in which a normally diploid cell or organism exhibits more than two sets of chromosomes; the term “aneuploidy” means any ploidy (more or less than the normal two sets of chromosomes).

The term “chromosomal structural abnormalities” refers to any change in the normal structure of a chromosome. Chromosomal structural abnormalities include, but are not limited to: duplications, deletions, translocations, inversions, and insertions.

The term “definitive endoderm” or “DE” refers to a multipotent cell that can differentiate into cells of the gut tube or organs derived from the gut tube. In accordance with certain embodiments, the definitive endoderm cells and cells derived therefrom are mammalian cells, and in a preferred embodiment, the definitive endoderm cells are human cells. In some embodiments, definitive endoderm cells express or fail to significantly express certain markers. In some embodiments, one or more markers selected from SOX17, CXCR4, MIXLI, GAT A4, FOXA2, GSC, FGF 17, VWF, CALCR, FOXQI, CMKORI, CER and CRIPI are expressed in definitive endoderm cells. In other embodiments, one or more markers selected from OCT4, HNF4A, alpha-fetoprotein (AFP), Thrombomodulin (TM), SPARC and SOX7 are not significantly expressed in definitive endoderm cells. Definitive endoderm cells do not express PDX-1.

As used herein, the phrase, “differentiable cell” or “differentiated cell” or “hES-derived cell” can refer to pluripotent, multipotent, oligopotent or even unipotent cells, as defined in detail below. In certain embodiments, the differentiable cells are pluripotent differentiable cells. In more specific embodiments, the pluripotent differentiable cells are selected from the group consisting of pluripotent stem cells, ICM/epiblast cells, primitive ectoderm cells, primordial germ cells, and teratocarcinoma cells. In one particular embodiment, the differentiable cells are mammalian pluripotent stem cells. In a more particular embodiment, the differentiable cells are human pluripotent stem cells. Certain embodiments also contemplate differentiable cells from any source within an animal, provided the cells are differentiable as defined herein. For example, differentiable cells can be harvested from embryos, or any primordial germ layer therein, from placental or chorion tissue, or from more mature tissue such as adult stem cells including, but not limited to adipose, bone marrow, nervous tissue, mammary tissue, liver tissue, pancreas, epithelial, respiratory, gonadal and muscle tissue. In specific embodiments, the differentiable cells are pluripotent stem cells. In other specific embodiments, the differentiable cells are adult stem cells. In still other specific embodiments, the stem cells are placental- or chorionic-derived stem cells.

As used herein, the term “differentiation” refers to the production of a cell type that is more differentiated than the cell type from which it is derived. The term therefore encompasses cell types that are partially and terminally differentiated. Similarly, “produced from hESCs,” “derived from hESCs,” “differentiated from hESCs,” “hES derived cell” and equivalent expressions refer to the production of a differentiated cell type from hESCs in vitro and in vivo.

The term “genomic instability” (also “genome instability or “genetic instability) refers to an increase in structural chromosomal alterations (deletions, amplifications, and translocations), numerical chromosomal aneuploidy, or mutations on DNA sequence within the genome of a cellular lineage.

The term “oncogenic potential” means the likelihood that a cell after its transplantation into a host will generate malignant tumors in the host. The term is applied for example to induced pluripotent stem cells, and to their propensity to generate malignant tumors upon differentiation and transplantation to an animal or human. Phenotypic traits such as genomic instability, impaired DNA damage response, reduced apoptosis response and reduced glucose metabolism indicate elevated oncogenic potential whether the iPSC has been derived from an aged donor or not.

The term “effective amount” of a factor or other active molecule means an amount effective to bring about a particular result. For example, in the case of ZSCAN10 or GLUT3 or exosome subunit supplementation (or GPX2 or GSS inhibition), an effective amount is that which brings about substantial restoration of apoptosis response, and/or DNA damage response and/or glucose metabolism defect or preserves genomic stability.

The term “reprogramming factors” refers to transcription factors i.e., proteins that alone, or in combination with other reprogramming factors, have the ability to reprogram differentiated somatic cells to cells to a pluripotent state.

The term “transcriptional pluripotency network” refers to a network of transcription factors involved in the transcriptional control of pluripotency in pluripotent stem cells (ESC). The present inventors have shown that ZSCAN10 is part of the “transcriptional pluripotency network” and should be supplemented in stem cells deficient in ZSCAN10 by comparison to Y-IPSC or ESC.

The term “mutagenic potential” refers to the potential or capacity of a substance to induce a change in the regulatory, protein-coding or other portions of a DNA sequence, increasing the frequency of mutations above a normal (background) level.

The term “young” used in connection with iPSC means iPSC derived from young donors, in case of mice up to 5 days old, in case of humans up to 16 years old and more generally to iPSC derived from donors that exhibit a “young” signature, e.g., slowing active growth stage to initiate the entry into fully grown adult stage.

The term “old” used in connection with iPSC means iPSC derived from aged donors, in case of mice older than 1.4 years old, in case of humans later than 50 years old, which begin to show age related degenerative diseases or states.

The term “substantial” used in the context of restoration, preservation recovery or rescue of glucose metabolism or DNA damage response, or apoptosis response, or genomic stability of A-iPSC denotes achievement of a state approximately or exactly the same as that of Y-iPSC and ESC. Finally, if the oxidation capacity of glutathione in A-iPSC is reduced (for example by supplementation of ZSCAN10 or by inhibition of GSS or GPX2) to be within the range from about 80% to about 120% of that of ESC or Y-iPSC, it is considered substantially restored.

The term “exosome” refers to the multi-protein exosome complex (or PM/Scl complex, often just called the exosome) capable of degrading various types of RNA (ribonucleic acid) molecules. Substrates of the exosome include messenger RNA, ribosomal RNA, and many species of small RNAs. Exosome comprises nine core subunits and two exonuclease co-factors.

The term “exosome subunit” refers to eleven components (listed in Table 3) of the exosome, comprising nine core subunits and two co-factors: EXOS1, EXOS2, EXOS3, EXOS4, EXOS5, EXOS6 EXOS7, EXOS8, EXOS9, EXOS10, and DIS3.

Unless otherwise required by context, singular terms shall include the plural. For example, “an exosome subunit” shall mean one or more exosome subunits.

In one embodiment iPSC are generated from various sources and utilized to generate insulin producing cells in vitro. In other embodiments the process is performed in vivo. Methods for generation of iPSCs from somatic cells involves forced expression of a set of polypeptides or induction factors (IFs). IFs currently known to the art include but are not limited to polypeptides encoded by the genes: c-Myc, Oct3/4, Sox2, and Klf4. In addition, small molecule compounds such as histone deacetylace inhibitors may be used or a combination of IFs and small molecules may be used to generate iPSCs. The somatic cells may be used directly, i.e., without culturing or passaging, in the referenced induction methods; or, the somatic cells may be cultured and/or passaged prior to their use in the referenced induction methods. The induced cells may be induced from the somatic cells of a postnatal donor or non-pluripotent donor as described in U.S. application Ser. No. 12/157,967, filed Jun. 13, 2008; First Inventor Kazuhiro Sakurada, which is herein incorporated by reference in its entirety. The induced stem cells may be generated from any cell-type including but not limited to those described.

IPSCs or iSCs may be used directly for differentiation or regenerative medicine. In other cases, iPSCs or iSCs may be stored by the regenerative medicine business, stem cell technology business or a third party. Alternatively, iPSCs or iSCs may be expanded using culturing methods described in U.S. application Ser. No. 12/157,967, filed Jun. 13, 2008; First Inventor Kazuhiro Sakurada, which is herein incorporated by reference in its entirety, prior to or after storage. iSCs may be stored in any manner which preserves their multipotent or pluripotent capabilities including cryogenic storage, and culturing. In some cases the donor, potential recipient of the iPSCs or derivatives thereof, or payee may be billed for generation and or delivery of iPSCs or differentiated cells or tissues. In some cases a kit may be marketed and sold which includes a means for generation of iPSCs. During the induction process, forced expression of certain polypeptides is carried out in cultured cells for a period of time, after which the induced cells are screened for a number of morphological and gene expression properties that characterize multipotent and pluripotent stem cells. Induced cells that meet these screening criteria may then be subcloned and expanded. In some cases, the cells to be induced may be cultured for a period of time prior to the induction procedure. Alternatively, the cells to be induced may be used directly in the induction process without a prior culture period. In some embodiments, the type of cell culture medium used is the same or very similar before, during, and after the induction process. In other cases, different cell culture media are used at different points. For example, one type of culture medium may be used directly before the induction process, while a second type of media is used during the induction process. At times, a third type of culture medium is used during the induction process. Cells may be cultured in medium supplemented with a particular serum. In some embodiments, the serum is fetal bovine serum (FBS). The serum can also be fetal calf serum (FCS). In some cases, the serum may be Human AB serum. Mixtures of serum may also be used, e.g. mixture of FBS and Human AB, FBS and FCS, or FCS and Human AB. Culture of cells may be carried out under a low serum culture conditions prior to, during, or following induction. A “low serum culture condition” refers to the use of a cell culture medium containing a concentration of serum ranging from 0% (v/v) (i.e., serum-free) to about 5% (v/v), e.g., 0% to 2%, 0% to 2.5%, 0% to 3%, 0% to 4%, 0% to 5%, 0.1% to 2%, 0.1% to 5%, 0.1%, 0.5%, 1%, 1.2%, 1.5%, 2%, 2.5%, 3%, 3.5%, or 4%. In some embodiments, the serum concentration is from about 0% to about 2%. In some cases, the serum concentration is about 2%. In some cases, the serum concentration is preferably 2% or less. In other embodiments, cells are cultured under a “high serum condition,” i.e., greater than 5% serum to about 20% serum, e.g., 6%, 7%, 8%, 10%, 12%, 15%, or 20%. Culturing under high serum conditions may occur prior to, during, and/or after induction.

Some representative media that the cells can be cultured in include: MAPC, FBM, ES, MEF-conditioned ES (MC-ES), and mTeSR™ (available, e.g., from StemCell Technologies, Vancouver, Canada), See Ludwig et al (2006), Nat Biotechnol, 24(2):185-187. In other cases, alternative culture conditions for growth of human ES cells are used, as described in, e.g., Skottman et al (2006), Reproduction, 132(5):691-698. In some embodiments, the cells are cultured in MAPC, FBM, MC-ES, or mTeSR™ prior to and/or during the introduction of induction factors to the cells; and the cells are cultured in MC-ES or mTeSR™ medium later in the induction process. MAPC (2% FBS) Medium may comprise: 60% Dulbecco's Modified Eagle's Medium-low glucose, 40% MCDB 201, Insulin Transferrin Selenium supplement, (0.01 mg/ml insulin; 0.0055 mg/ml transferrin; 0.005 μg/ml sodium selenite), 1× linolenic acid albumin (1 mg/ml albumin; 2 moles linoneic acid/mole albumin), 1 nM dexamethasone, 2% fetal bovine serum, 1 nM dexamethasone, 10-4 M ascorbic acid, and 10 pg/ml gentamycin. FBM (2% FBS) Medium may comprise: MCDB202 modified medium, 2% fetal bovine serum, 5 μg/ml insulin, 50 mg/ml gentamycin, and 50 ng/ml amphotericin-B. ES Medium may comprise: 40% Dulbecco's Modified Eagle's Medium (DMEM) 40% F12 medium, 2 mM L-glutamine, 1× non-essential amino acids (Sigma, Inc., St. Louis, Mo.), 20% Knockout Serum Replacement™ (Invitrogen, Inc., Carlsbad, Calif.), and 10 μg/ml gentamycin.

MC-ES medium may be prepared as follows. ES medium is conditioned on mitomycin C-treated murine pluripotent fibroblasts (MEFs), harvested, filtered through a 0.45-μM filter, and supplemented with about 0.1 mM B mercaptoethanol, about 10 ng/ml bFGF or FGF-2, and, optionally, about 10 ng/ml activin A. In some cases, irradiated MEFs are used in place of the mitomycin C-treated MEFs. When either low or high serum conditions are used for culturing the cells, one or more growth factors such as fibroblast growth factor (FGF)-2; basic FGF (bFGF); platelet-derived growth factor (PDGF), epidermal growth factor (EGF); insulin-like growth factor (IGF); or insulin can be included in the culture medium. Other growth factors that can be used to supplement cell culture media include, but are not limited to one or more: Transforming Growth Factor □-1 (TGF □-1), Activin A, Noggin, Brain-derived Neurotrophic Factor (BDNF), Nerve Growth Factor (NGF), Neurotrophin (NT)-1, NT-2, or NT 3. In some cases, one or more of such factors is used in place of the bFGF or FGF-2 in the MC-ES medium or other cell culture medium. In some cases, the concentration of growth factors in the culture media described (e.g., MAPC, FBM, MC-ES, mTeSR™) is from about 2 ng/ml to about 20 ng/ml, e.g., about 2 ng/ml, 3 ng/ml, 4 ng/ml, 5 ng/ml, 6 ng/ml, 7 ng/ml, 8 ng/ml, 10 ng/ml, 12 ng/ml, 14 ng/ml, 15 ng/ml, 17 ng/ml, or 20 ng/ml. In some embodiments, the concentration of bFGF or FGF2 is from about 2 ng/ml to about 5 ng/ml; from about 5 ng/ml to about 8 ng/ml; from about 9 ng/ml to about 11 ng/ml; from about 11 ng/ml to about 15 ng/ml; or from about 15 ng/ml to about 20 ng/ml. The growth factors may be used alone or in combination. For example, FGF-2 may be added alone to the medium; in another example, both PDGF and EGF are added to the culture medium. In some examples, following initiation of the forced expression of genes or polypeptides (e.g., immediately after a retroviral infection period) in cells, the “induced cells” are maintained in MC-ES medium as described herein.

In some embodiments, cells are maintained in the presence of a rho, or rho-associated, protein kinase (ROCK) inhibitor to reduce apoptosis. In some cases, an inhibitor of Rho associated kinase is added to the culture medium. For example, the addition of Y-27632 (Calbiochem; water soluble) or Fasudil (HA1077: Calbiochem), an inhibitor of Rho associated kinase (Rho associated coiled coil-containing protein kinase) may be used to culture the human pluripotent and multipotent stem cells of the present invention. In some cases the concentration of Y-27632 or Fasudil, is from about 5 μM to about 20 μM, e.g., about 5 M, 10 μM, 15 μM, or 20 μM. The cells may be cultured for about 1 to about 12 days e.g., 2 days, 3 days, 4.5 days, 5 days, 6.5 days, 7 days, 8 days, 9 days, 10 days, or any other number of days from about 1 day to about 12 days prior to undergoing the induction methods described herein. In some cases, the induced cells are cultured in complete ES medium in a 37□C, 5% CO2 incubator, with medium changes about every 1 to 2 days. In some embodiments, induced the induced cells are cultured and observed for about 14 days to about 40 days, e.g., 15, 16, 17, 18, 19, 20, 23, 24, 27, 28, 29, 30, 31, 33, 34, 35, 36, 37, 38 days, or any other period from about 14 days to about 40 days prior to identifying and selecting clones comprising “induced cells” based on morphological characteristics. Morphological characteristics for identifying induced cell clones include, but are not limited to, a small cell size with a high nucleus-to-cytoplasm ratio; formation of small monolayer colonies within the space between parental cells (e.g., between fibroblasts). The cells may be plated at a cell density of about 1×103 cells/cm2 to about 1×104 cells/cm2, e.g., 2×103 cells/cm2, 3.5×103 cells/cm2, 6×103 cells/cm2, 7×103 cells/cm2, 9×103 cells/cm2, or any other cell density from about 1×103 cells/cm2 to about 1×104 cells/cm2. The cells can be plated and cultured directly on tissue culture-grade plastic. Alternatively, cells are plated and cultured on a coated substrate, e.g., a substrate coated with fibronectin, gelatin, matrigel™, collagen, or laminin. Suitable cell culture vessels include, e.g., 35 mm, 60 mm, 100 mm, and 150 mm cell culture dishes, 6-well cell culture plates, and other size-equivalent cell culture vessels. In some cases, the cells are cultured with feeder cells. For example, the cells may be cultured on a layer, or carpet, of MEFs.

Media with low concentrations of serum may be particularly useful to enrich for undifferentiated stem cells. The undifferentiated cells cultured under low serum conditions may or may not share certain properties with MSCs, MAPCs, and/or MIAMI cells. Differences in phenotype may be due, in part, to culture methods used to obtain MSCs, MAPCs and MIAMI cells. For example, MSCs are often obtained by isolating the non-hematopoeitic cells (e.g., interstitial cells) adhering to a plastic culture dish when tissue, e.g., bone marrow, fat, muscle, or skin etc., is cultured in a culture medium containing a high-concentration serum (5% or more). However, even under these culture conditions, a very small number of undifferentiated cells can be maintained, especially if the cells were passaged under certain culture conditions (e.g., low passage number or low-density culturing). In some embodiments, in order to culture and grow human pluripotent stem cells induced from the undifferentiated stem cells of the present invention present in a human postnatal tissue, it is preferred that the cells are subcultured every 5 to 7 days in a culture medium containing the additives described herein on a MEF-covered plastic culture dish or a matrigel-coated plastic culture dish. In some cases, the cells may be cultured at a low density, which may be accomplished by splitting the cells from about 1:6 to 1:3 or by plating the cells at 103 cells/cm2 to 3×104 cells/cm2. Primary culture ordinarily occurs immediately after the cells are isolated from a donor, e.g., human. The primary cells can be subjected to a second subculture, a third subculture, a fourth subculture, and greater than four subcultures. A “second” subculture describes primary culture cells subcultured once, a “third” subculture describes primary cultures subcultured twice, a “fourth” subculture describes primary cells subcultured three times, etc. The culture techniques described herein may generally include culturing from the period between the primary culture and the fourth subculture, but other culture periods may also be employed. Preferably, cells are cultured from primary culture to second subculture.

Inducing a cell to become multipotent or pluripotent can be accomplished in numerous ways. In some embodiments, the methods for induction of pluripotency or multipotency in one or more cells include forcing expression of a set of induction factors (IFs). In some cases, the set of IFs includes one or more: an Oct3/4 polypeptide, a Sox2 polypeptide, a Klf4 polypeptide, or a c-Myc polypeptide. In some cases, the set does not include a c-Myc polypeptide. For example, the set of IFs can include: an Oct3/4 polypeptide, a Sox2 polypeptide, and a Klf4 polypeptide, but not a c-Myc polypeptide. In some cases, the set of IFs does not include polypeptides that might increase the risk of cell transformation. In some cases, the set may include a c-Myc polypeptide. In certain cases, the c-Myc polypeptide is a constitutively active variant of c-Myc. In some instances, the set includes a c-Myc polypeptide capable of inducible activity, e.g., a c-Myc-ER polypeptide,

In other cases, the set of IFs may include: an Oct3/4 polypeptide, a Sox2 polypeptide, and a Klf4 polypeptide, but not a TERT polypeptide, a SV40 Large T antigen polypeptide, HPV16 E6 polypeptide, a HPV16 E7 polypeptide, or a Bmil polypeptide. In some cases, the set of IFs does not include a TERT polypeptide. In some cases, the set of IFs does not include a SV40 Large T antigen. In other cases, the set of IFS does not include a HPV 16 E6 polypeptide or a HPV 16 E7 polypeptide.

In some cases, the set of IFs includes three IFs, wherein two of the three IFs are an Oct3/4 polypeptide and a Sox2 polypeptide. In other cases, the set of IFs includes two IFs, wherein the two polypeptides are a c-Myc polypeptide and a Sox2 polypeptide In some cases, the set of induction factors is limited to October 3/4, Sox2, and Klf4 polypeptides. In other cases, the set of induction factors may be limited to a set of four IFs: an Oct3/4 polypeptide, a Sox2 polypeptide, a Klf4 polypeptide, and a c-Myc polypeptide. A set of IFs may include IFs in addition to an October 3/4, a Sox2, and a Klf4 polypeptide. Such additional IFs include, but are not limited to Nanog, TERT, LIN28, CYP26A1, GDF3, FoxD3, Zfp42, Dnmt3b, Ecat1, and Tcl1 polypeptides. In some cases, the set of additional IFs does not include a c Myc polypeptide. In some cases, the set of additional IFs does not include polypeptides that might increase the risk of cell transformation. Forced expression of IFs may be maintained for a period of at least about 7 days to at least about 40 days, e.g., 8 days, 9 days, 10 days, 11 days, 12 days, 13 days, 14 days, 15 days, 16 days, 17 days, 18 days, 19 days, 20 days, 21 days, 25 days, 30 days, 33 days, or 37 days. In a first step, a cell population (the starting cell population) comprising at least one cell capable of differentiation is provided. In some embodiments, the cell capable of differentiation is a pancreatic progenitor cell expressing PDX1 and NKX6.1.

In some embodiments, the starting cell population comprises at least 5% pancreatic progenitor cells, such as at least 10% pancreatic progenitor cells, such as at least 15% pancreatic progenitor cells, such as at least 20% pancreatic progenitor cells, such as at least 25% pancreatic progenitor cells, such as at least 30% pancreatic progenitor cells, such as at least 35% pancreatic progenitor cells, such as at least 40% pancreatic progenitor cells, such as at least 45% pancreatic progenitor cells, such as at least 50% pancreatic progenitor cells, such as at least 55% pancreatic progenitor cells, such as at least 60% pancreatic progenitor cells, such as at least 65 pancreatic progenitor cells, such as at least 70% pancreatic progenitor cells, such as at least 75% pancreatic progenitor cells, such as at least 80% pancreatic progenitor cells, such as at least 85% pancreatic progenitor cells, such as at least 90% pancreatic progenitor cells, such as at least 95% pancreatic progenitor cells. In order to determine the fraction of progenitor cells comprised in a cell population, for example in the starting population, methods known in the art can be employed, such as, but not limited to, immunostaining or flow cytometry methods.

Without being bound by theory, the percentage of pancreatic progenitor cells in the starting cell population can be estimated by the expression of GP2. Thus in some embodiments, the starting cell population comprises at least 5% cells expressing GP2, such as at least 10% cells expressing GP2, such as at least 15% cells expressing GP2, such as at least 20% cells expressing GP2, such as at least 25% cells expressing GP2, such as at least 30% cells expressing GP2, such as at least 35% cells expressing GP2, such as at least 40% cells expressing GP2, such as at least 45% cells expressing GP2, such as at least 50% cells expressing GP2, such as at least 55% cells expressing GP2, such as at least 60% cells expressing GP2, such as at least 65% cells expressing GP2, such as at least 70% cells expressing GP2, such as at least 75% cells expressing GP2, such as at least 80% cells expressing GP2, such as at least 85% cells expressing GP2, such as at least 90% pancreatic progenitor cells, such as at least 95% pancreatic progenitor cells. GP2 expression can be determined by methods known in the art, such as immunostaining methods, flow cytometry methods or quantitative measurements of transcription levels. Likewise, without being bound by theory, the percentage of PDX1+NKX6.1+ cells in the starting cell population can be estimated by the expression of GP2. Thus in some embodiments, the starting cell population comprises at least 5% cells expressing GP2, such as at least 10% cells expressing GP2, such as at least 15% cells expressing GP2, such as at least 20% cells expressing GP2, such as at least 25% cells expressing GP2, such as at least 30% cells expressing GP2, such as at least 35% cells expressing GP2, such as at least 40% cells expressing GP2, such as at least 45% cells expressing GP2, such as at least 50% cells expressing GP2, such as at least 55% cells expressing GP2, such as at least 60% cells expressing GP2, such as at least 65% cells expressing GP2, such as at least 70% cells expressing GP2, such as at least 75% cells expressing GP2, such as at least 80% cells expressing GP2, such as at least 85% cells expressing GP2, such as at least 90% pancreatic progenitor cells, such as at least 95% pancreatic progenitor cells. GP2 expression can be determined by methods known in the art, such as immunostaining methods, flow cytometry methods or quantitative measurements of transcription levels.

In some embodiments, the cell population may be derived or isolated from an individual, such as, but not limited to, a mammal, for example a human. In some embodiments, the cells capable of differentiation are pluripotent stem cells, for example human pluripotent stem cells (hPSCs). hPSCs include human induced pluripotent stem cells (hiPSCs and naïve human stem cells (NhSCs). In one embodiment, the starting cell population is obtained from a pancreas, including a foetal pancreas or an adult pancreas. In one aspect, the pancreas is from a mammal, such as a human. In another embodiment, the starting cell population is a somatic cell population. In some embodiments, the starting cell population comprises at least one pancreatic progenitor cell expressing PDX1 and NKX6.1 and is obtained from a somatic cell population. In a further aspect of the invention, the somatic cell population has been induced to de-differentiate into an pluripotent-like stem cell (ESC, e.g. a pluripotent stem cell, or hESCs for human ESCs). Such dedifferentiated cells are also termed induced pluripotent stem cells (IPSCs, or hIPSCs for human IPSCs). In yet another embodiment, the starting cell population is ESCs or hESCs. In one embodiment, the starting cell population is obtained from ESCs or hESCs. In some embodiments, the starting cell population is a population of pluripotent stem cells such as ESC like-cells. In some embodiments, a cell population comprising at least one pancreatic progenitor cell may be obtained by methods known in the art, before steps viii) and ix) as described herein are performed. For example, differentiation can be induced in embryoid bodies and/or in monolayer cell cultures or a combination thereof. In one aspect of the invention, the starting cell population is of mammalian origin. In one aspect of the invention, the starting cell population is of human origin.

In one aspect of the invention, the starting cell population is obtained from one or more donated pancreases. The methods described herein are not dependent on the age of the donated pancreas. Accordingly, pancreatic material isolated from donors ranging in age from embryos to adults can be used. Once a pancreas is harvested from a donor, it is typically processed to yield individual cells or small groups of cells for culturing using a variety of methods. One such method calls for the harvested pancreatic tissue to be cleaned and prepared for enzymatic digestion. Enzymatic processing is used to digest the connective tissue so that the parenchyma of the harvested tissue is dissociated into smaller units of pancreatic cellular material. The harvested pancreatic tissue is treated with one or more enzymes to separate pancreatic cellular material, substructures, and individual pancreatic cells from the overall structure of the harvested organ. Collagenase, DNAse, lipase preparations and other enzymes are contemplated for use with the methods disclosed herein. Isolated source material can be further processed to enrich for one or more desired cell populations prior to performing the present methods. In some aspects unfractionated pancreatic tissue, once dissociated for culture, can also be used directly in the culture methods of the invention without further separation. However, unfractionated pancreatic tissue, once dissociated for culture, can also be used directly in the culture methods of the invention without further separation, and will yield the intermediate cell population. In one embodiment the isolated pancreatic cellular material is purified by centrifugation through a density gradient (e. g., Nycodenz, Ficoll, or Percoll). The mixture of cells harvested from the donor source will typically be heterogeneous and thus contain alpha cells, beta cells, delta cells, ductal cells, acinar cells, facultative progenitor cells, and other pancreatic cell types. A typical purification procedure results in the separation of the isolated cellular material into a number of layers or interfaces. Typically, two interfaces are formed. The upper interface is islet-enriched and typically contains 10 to 100% islet cells in suspension. The second interface is typically a mixed population of cells containing islets, acinar, and ductal cells. The bottom layer is the pellet, which is formed at the bottom of the gradient. This layer typically contains primarily acinar cells, some entrapped islets, and some ductal cells. Ductal tree components can be collected separately for further manipulation. The cellular constituency of the fractions selected for further manipulation will vary depending on which fraction of the gradient is selected and the final results of each isolation. When islet cells are the desired cell type, a suitably enriched population of islet cells within an isolated fraction will contain at least 10% to 100% islet cells. Other pancreatic cell types and concentrations can also be harvested following enrichment. For example, the culture methods described herein can be used with cells isolated from the second interface, from the pellet, or from other fractions, depending on the purification gradient used. In one embodiment, intermediate pancreatic cell cultures are generated from the islet-enriched (upper) fraction. Additionally, however, the more heterogeneous second interface and the bottom layer fractions that typically contain mixed cell populations of islets, acinar, and ductal cells or ductal tree components, acinar cells, and some entrapped islet cells, respectively, can also be used in culture. While both layers contain cells capable of giving rise to the enriched pancreatic progenitor cell population described herein, each layer may have particular advantages for use with the disclosed methods. In some embodiments, the cell population comprising at least one pancreatic progenitor cell is analysed to identify whether at least one of the cells of the starting population expresses markers characteristic of the pancreatic endocrine lineage and selected from the group consisting of NGN3, NEUROD, ISL1, PDX1, NKX6.1, NKX2.2, MAFA, MAFB, ARX, BRN4, PAX4 and PAX6, GLUT2, INS, GCG, SST, pancreatic poly-peptide (PP). In some embodiments markers characteristic of the pancreatic endocrine lineage are selected from the group consisting of PDX1 and NKX6.1. In one embodiment, a pancreatic endocrine cell is capable of expressing at least one of the following hormones: insulin, glucagon, somatostatin, and PP. In some embodiments, a pancreatic endocrine cell is capable of expressing at least one of the following hormones: insulin, glucagon, somatostatin, PP and ghrelin. Suitable for use in the present invention is a cell that expresses at least one of the markers characteristic of the pancreatic endocrine lineage. In one aspect of the present invention, a cell expressing markers characteristic of the pancreatic endocrine lineage is a pancreatic endocrine cell. The pancreatic endocrine cell may be a pancreatic hormone expressing cell. Alternatively, the pancreatic endocrine cell may be a pancreatic hormone secreting cell. In one embodiment, the pancreatic endocrine cell is a cell expressing markers characteristic of the beta cell lineage. A cell expressing markers characteristic of the beta cell lineage expresses PDX1 and may further express at least one of the following transcription factors: NGN3, NKX2-2, NKX6.1, NEUROD, ISL1, FOXA2, MAFA, PAX4, and PAX6. In one embodiment, a cell expressing markers characteristic of the beta cell lineage is a beta cell. In one embodiment, the pancreatic endocrine cell is a cell expressing the marker NKX6.1. In another aspect of the invention, the pancreatic endocrine cell is a cell expressing the marker PDX1. In a further aspect of the invention, the pancreatic endocrine cell is a cell expressing the markers NKX6.1 and PDX1. PDX1 is homeodomain transcription factor implicated in pancreas development. Pax-4 is a beta cell specific factor and Pax-6 is a pancreatic islet cell (specific) transcription factor; both are implicated in islet development. Hnf-3 beta (also known as FoxA2) belongs to the hepatic nuclear factor family of transcription factors, which is characterized by a highly conserved DNA binding domain and two short carboxy-terminal domains. NeuroD is basic helix-loop-helix (bHLH) transcription factor implicated in neurogenesis. Ngn3 is a member of the neurogenin family of basic loop-helix-loop transcription factors. NKX2-2 and NKX6.1 as used herein are members of the Nkx transcription factor family. Islet-1 or ISL-1 is a member of the LIM/homeodomain family of transcription factors, and is expressed in the developing pancreas. MAFA is a transcription factor expressed in the pancreas, and controls the expression of genes involved in insulin biosynthesis and secretion. NKX6.1 and PDX1 are co-expressed with PTF1a in the early pancreatic multipotent cell that can develop into all cell types found in the adult pancreas (e.g., acinar, ductal, and endocrine cells). Within this cell population cells that also transiently express NGN3 are found. Once a cell expresses or has expressed NGN3 it will be part of the endocrine lineage, giving rise to endocrine cells (one type being the insulin producing beta cell) that will later form the Islets of Langerhans. In the absence of NGN3 no endocrine cells form during pancreas development. As development progress NKX6.1 and PDX1 are co-expressed in the more central domain of the pancreas, which now becomes devoid of PTF1a expression and the NKX6.1 and PDX1 positive cells can no longer give rise to acinar cells. Within this NKX6.1 and PDX1 positive cell population a significant number of cells transiently co-express NGN3, marking them for the endocrine lineage like earlier in development. In one embodiment, the cells comprised in the starting cell population are derived from cells capable of differentiation. In a specific embodiment, the cells capable of differentiation are human pluripotent stem cells. In some embodiments, the cells capable of differentiation are selected from the group consisting of human iPS cells (hIPSCs), human ES cells (hESCs) and naive human stem cells (NhSCs). The cells capable of differentiation may be derived from cells isolated from an individual.

CDKN1a, also dubbed P21, and CDKN2a, also P16, are cell cycle specific genes. CDKN1a (cyclin-dependent kinase inhibitor 1 or CDK-interacting protein 1), is a cyclin-dependent kinase inhibitor that inhibits the complexes of CDK2 and CDK1. CDKN1 thus functions as a regulator of cell cycle progression at G1 and S phase. CDKN2a (cyclin-dependent kinase inhibitor 2A, multiple tumor suppressor 1) is a tumor suppressor protein. It plays an important role in cell cycle regulation by decelerating cells progression from G1 phase to S phase, and therefore acts as a tumor suppressor that is implicated in the prevention of cancers. The inventors have surprisingly found that inactivation of CDKN1a or CDKN2a in the starting cell population facilitates entry of the cell population in a replicating state corresponding to the G2/M phase, in particular when the starting cell population is PDX1 expressing pancreatic progenitor cells. Inactivation of CDKN1a or CDKN2a in the starting cell population may also facilitate entry of the cell population in the S phase. Thus inactivation of CDKN1a or CDKN2a may be useful for obtaining mature beta cells from expanded pancreatic progenitor cells. In some embodiments, expression of CDKN1a and/or CDKN2a in the starting cell population is inactivated. In some embodiments, the starting cell population is a population of pancreatic progenitor cells expressing PDX1. The starting cell population may also be any of the populations described above. The skilled person knows how to inactivate expression of CDKN1a and/or CDKN2a. This may be done for instance by mutating or deleting the corresponding genes, by known gene editing methods. Alternatively, silencing means may be employed such as siRNA in order to prevent expression of CDKN1a and/or CDKN2a. Alternatively, inhibitors preventing correct function of CDKN1a and/or CDKN2a may be used. In one embodiment of the invention, exosomes are utilized to induce generation of pluripotent stem cells in vitro or in vivo. When we use the term “exosome”, we refer to a membrane vesicle that is extracellularly secreted from a cell or has a membrane structure composed of a lipid-bilayer present in the cell, and the exosome exists in the body fluid of almost all eukaryotes. Exosomes have a diameter of approximately 30-300 nm, and exosomes are released from cells when multivesicular bodies are fused to cell membranes, or released directly from cell membranes. Exosomes are well known to serve to transport intracellular biomolecules, such as proteins, bioactive lipids, and RNA (miRNA), so as to perform a functional role of mediating coagulation, cell-cell communication, and cellular immunity. In the present invention, the concept of the exosomes encompasses microvesicles. The marker proteins of exosomes are known to be CD63, CD81, or the like, and besides, are known to be proteins, for example, cell surface receptors such as EGFR, signaling-related molecules, cell adhesion-related proteins, MSC-associated antigens, heat shock proteins, vesiculation-related Alix. In an embodiment of the present invention, the pluripotent stem cell derived exosomes are obtained from cultures of said cells, typically cultures are performed in liquid media, for example, Dulbecco's modification of Eagle's medium (DMEM), a mixture of DMEM and F12, Eagle's minimum essential medium (Eagle's MEM), α-MEM, Iscove's MEM, 199 medium, CMRL 1066, RPMI 1640, F12, F10, Way-mouth's MB752/1, McCoy's 5A, MCDB series, and the like may be used. In some embodiments stimulation with interferon gamma or other cytokines is performed. In an embodiment of the present invention, the interferon-gamma is contained in the cell culture medium at a concentration of 1-100 ng/ml. In another embodiment of the present invention, the interferon-gamma is contained in the cell culture medium at a concentration of 1-90 ng/ml, 1-80 ng/ml, 1-70 ng/ml, 1-60 ng/ml, 1-50 ng/ml, 1-40 ng/ml, 1-30 ng/ml, 10-90 ng/ml, 10-80 ng/ml, 10-70 ng/ml, 10-60 ng/ml, 10-50 ng/ml, 10-40 ng/ml, and 10-30 ng/ml. In an embodiment of the present invention, the culturing is performed for 6-48 hours. In another embodiment of the present invention, the culturing is performed for 6-42 hours, 6-36 hours, 6-30 hours, 6-27 hours, 12-48 hours, 12-42 hours, 12-36 hours, 12-30 hours, 12-27 hours, 18-48 hours, 18-42 hours, 18-36 hours, 18-30 hours, 18-27 hours, 21-48 hours, 21-42 hours, 21-36 hours, 21-30 hours, or 21-27 hours.

The present invention relates, in part, to the transcriptional regulations that are critical to induce B-cell differentiation from ES cell-derived endoderm. For example, the combination of Pdx1 and Ngn3 induces pancreatic endocrine genes as well as B-cell-related transcriptional factors such as Pax4, Pax6, Isl1 and Nkxx2.2. Other pancreas-related proteins such, as C-peptide and insulin, can be detected by immunohistochemistry in these cells. In addition, these cells process and secrete insulin and respond to various insulin secretagogues.

The present invention provides pancreatic endocrine progenitor cells and methods for producing pancreatic endocrine progenitor cells from pluripotent stem cells or from induced Pluripotent Stem (iPS) cells. The endocrine progenitor cells are useful to identify agents that modulate pancreatic endocrine function, to identify agents that affect cell growth and differentiation, to identify genes involved in pancreatic tissue development and to generate differentiated cells and tissues for cell replacement therapies.

The invention is based, in part, on the discovery that overexpression of Pdx1 and Ngn3 can induce differentiation of pluripotent stem cell derived endoderm to a pancreatic endocrine cell fate. Forced expression of Pdx1 results in upregulation of pancreas-related genes such as insulin 1 (ins1) and insulin 2 (ins2) at day 20 of differentiation. Forced expression of Pdx1 and Ngn3 dramatically increases ins1 mRNA and at an earlier time, day 9, compared to Pdx alone. Forced expression of additional genes may further differentiation toward specific pancreatic endocrine cells. For, example, forced expression of Pdx1, Ngn3 and MafA may further induce differentiation of endoderm to a B cell lineage. As with pluripotent stem cell derived endoderm, Pdx1 and Ngn3 overexpression may induce differentiation of iPS cell derived endoderm to a pancreatic endocrine cell fate. The present invention provides pluripotent stem cells modified to overexpress Pdx1 and Ngn3. In some aspects, the invention provides iPS cells modified to overexpress Pdx1 and Ngn3. Expression of Pdx1 and Ngn3 may be simultaneous or expression of Pdx1 and Ngn3 may be sequential. In some aspects of the invention, Pdx1 and Ngn3 are under the control of one or more inducible promoters. The use of inducible promoters may facilitate the temporal expression of Pdx1 and Ngn3 in ES cells or iPS cells. For example, before differentiation into endoderm, it may be desired to minimize expression of Pdx1 and Ngn3. Inducible promoters generally exhibit low activity in the absence of inducer. Following differentiation of ES cells or iPS cells to endoderm, overexpression of Pdx1 and Ngn3 may be induced to direct differentiation of the endoderm to a pancreatic endocrine progenitor fate. Timing of induction of Pdx1 and Ngn3 can be used to optimize differentiation of endoderm to pancreatic endocrine progenitor cells.

In some aspects of the invention, Pdx1 may be under the control of one inducible promoter and Ngn3 may be under the control of a different inducible promoter. In this case, expression of Pdx1 and Ngn3 may be controlled temporally relative to one another by controlled induction of the different inducible promoters. In some aspects of the invention, Pdx1 and Ngn3 are under the control of the same inducible promoter. In this case, the pdx1 and ngn3 genes may be linked in an expression cassette. For example, the pdx1 and ngn3 genes can be linked in one expression cassette through the use of an Internal Ribosome Entry Site (IRES). In some aspects, the invention provides pluripotent cells modified with a pdx1-IRES-ngn3 expression cassette operably linked to a tetracycline-inducible promoter. In some cases, a Tet-pdx1-IRES-ngn3 expression cassette is stably introduced into the cells. In some cases, a Tet-pdx1-IRES-ngn3 expression cassette is transiently introduced into cells. The invention provides cells modified to express a reporter molecule used to monitor differentiation of cells to pancreatic endocrine progenitor cells. In some aspects, the invention provides iPS cells modified to express a reporter molecule used to monitor differentiation of iPS cells to pancreatic endocrine progenitor cells. The reporter molecule is operably linked to a promoter that is expressed in pancreatic endocrine progenitor cells or derivatives thereof but not expressed in primitive endoderm. In some aspects of the invention, the reporter molecule is B-lactamase (BLA). In some aspects of the invention, the promoter expressed in pancreatic endocrine progenitor cells or derivatives thereof but not expressed in primitive endocrine cells is the promoter controlling the expression of a pancreatic endocrine hormone. For example, the promoter may be, but is not limited to, an insulin 1 promoter, an insulin 2 promoter, a glucagon promoter, a somatostatin promoter, a pancreatic polypeptide promoter and a ghrelin/obestatin preprohormone promoter. In some aspects of the invention, ES cells are modified to express BLA under the control of the ins1 promoter. In some cases, an Ins1-BLA expression cassette is stably introduced into the ES cells. In some cases, an Ins1-BLA expression cassette is transiently introduced into ES cells.

The invention provides iPS cells that are modified to overexpress Pdx1, Ngn3 and MafA. Expression of Pdx1, Ngn3 and MafA may be simultaneous or expression of Pdx1, Ngn3 and MafA may be sequential. In some aspects of the invention, Pdx1, Ngn3 and MafA are under the control of one or more inducible promoters. Timing of induction of Pdx1, Ngn3 and MafA can be used to optimize differentiation of endoderm to pancreatic endocrine progenitor cells and to primitive beta-islet cells. In some aspects of the invention, Pdx1, Ngn3 and MafA may be under the control of different inducible promoters. In this case, expression of Pdx1, Ngn3 and MafA may be controlled temporally relative to one another by controlled activation of the different inducible promoters. In some aspects of the invention, Pdx1 and Ngn3 are under the control of the same inducible promoter, as described above, and MafA is under the control of a different promoter. In some cases, expression of MafA is controlled by an inducible promoter. In some cases, MafA is controlled by a constitutive promoter. In some aspects, the invention provides ES cells or iPS cells modified to overexpress Pdx1, Ngn3 and MafA and modified to express a reporter molecule under the control of a promoter expressed in pancreatic endocrine progenitor cells or derivatives thereof but not expressed in primitive endoderm. The invention provides methods to produce pluripotent stem cells modified to overexpress Pdx1 and Ngn3. In some aspects of the invention, nucleic acid encoding pdx1 and ngn3 genes are introduced into ES cells. In some cases the nucleic acids encoding pdx1 and ngn3 genes are stably introduced into the ES cells. In some cases the nucleic acid encoding pdx1 and ngn3 genes are transiently introduced into the ES cells. In some aspects, the invention provides methods to produce ES cells modified to overexpress Pdx1 and Ngn3 where the pdx1 and ngn3 genes are integrated into the ES genome. In some cases, the pdx1 and ngn3 genes are targeted to specific sites in the ES genome. For example, the pdx1 and ngn3 genes may be targeted to the HPRT locus or to the ROSA26 locus. Targeting can be accomplished using methods known in the art; for example, homologous recombination or through the use of a cre-lox recombination system. In some aspects, the invention provides methods to produce pluripotent stem cells modified to overexpress Pdx1, Ngn3 and MafA. In some aspects of the invention, nucleic acid encoding pdx1, ngn3 and mafA genes are introduced into ES cells. In some cases, the nucleic acids encoding one or more of pdx1, ngn3 and mafA genes are stably introduced into the ES cells. In some cases, the nucleic acids encoding one or more of pdx1, ngn3 and mafA genes are transiently introduced into the ES cells. In some aspects, the invention provides methods to produce ES cells modified to overexpress Pdx1, Ngn3 and MafA where the pdx1, ngn3 and mafA genes are integrated into the ES genome. In some cases, the pdx1, ngn3 and mafA genes are targeted to specific sites in the ES genome. For example, the pdx1, ngn3 and mafA genes may be targeted to the HPRT locus or to the ROSA26 locus. Targeting can be accomplished using methods known in the art; for example, homologous recombination or through the use of a cre-lox recombination system. The invention provides methods to produce iPS cells modified to overexpress Pdx1 and Ngn3. In some aspects of the invention, nucleic acid encoding pdx1 and ngn3 genes are introduced into iPS cells. In some cases the nucleic acids encoding pdx1 and ngn3 genes are stably introduced into the iPS cells. In some cases, nucleic acids encoding pdx1 and ngn3 genes are introduced to differentiated cells before induction to pluripotent stem cells. In some cases, nucleic acids encoding pdx1 and ngn3 are introduced to iPS cells after reprogramming of differentiated cells. In some cases, nucleic acids encoding pdx1 and ngn3 are introduced to cells during the reprogramming process. In some cases the nucleic acid encoding pdx1 and ngn3 genes are transiently introduced into the iPS cells. In some aspects, the invention provides methods to produce iPS cells modified to overexpress Pdx1 and Ngn3 where the pdx1 and ngn3 genes are integrated into the iPS genome. In some cases, the pdx1 and ngn3 genes are targeted to specific sites in the iPS genome. Targeting can be accomplished using methods known in the art; for example, homologous recombination or through the use of a cre-lox recombination system. In some aspects, the invention provides methods to produce iPS cells modified to overexpress Pdx1, Ngn3 and MafA. In some aspects of the invention, nucleic acid encoding pdx1, ngn3 and mafA genes are introduced into iPS cells. In some cases, the nucleic acids encoding one or more of pdx1, ngn3 and mafA genes are stably introduced into the iPS cells. In some cases, nucleic acids encoding pdx1, ngn3 and mafA genes are introduced to differentiated cells before induction to pluripotent stem cells. In some cases, nucleic acids encoding pdx1, ngn3 and mafA are introduced to iPS cells after reprogramming of differentiated cells. In some cases, nucleic encoding pdx1 and ngn3 and mafA are introduced to cells during the reprogramming process. In some cases, the nucleic acids encoding one or more of pdx1, ngn3 and mafA genes are transiently introduced into the iPS cells. In some aspects, the invention provides methods to produce iPS cells modified to overexpress Pdx1, Ngn3 and MafA where the pdx1, ngn3 and mafA genes are integrated into the iPS genome. In some cases, the pdx1, ngn3 and mafA genes are targeted to specific sites in the iPS genome. Targeting can be accomplished using methods known in the art; for example, homologous recombination or through the use of a cre-lox recombination system.

The invention provides methods to generate pancreatic endocrine progenitor cells and derivatives of pancreatic progenitor cells by forced expression of Pdx1 and Ngn3 in endoderm. A generalized scheme of differentiation of an endoderm precursor cells (e.g. definitive endoderm) to a variety of pancreatic cells in provided in FIG. 1. In some aspects of the invention, pluripotent cells such as ES cells or iPS cells are induced to form definitive endoderm. Overexpression of Pdx1 may lead to the formation of pancreatic progenitor cells. Overexpression of Pdx1 and Ngn3 may lead to the formation of pancreatic endocrine progenitor cells. Pancreatic endocrine progenitor cells may differentiate into cells secreting pancreatic endocrine hormones following expression of genes associated with a particular differentiation pathway. For example, overexpression of MafA in pancreatic endocrine progenitor cells may lead to the generation of primitive beta-islet cells. The invention provides methods of producing pancreatic endocrine progenitor cells from pluripotent stem cells. In some aspects, ES cells are first allowed to begin differentiation. Cells are then induced to form definitive endoderm. In some cases, cells are induced to form definitive endoderm by incubating cells in the presence of activin A. Pancreatic endocrine progenitor cells are then induced by overexpression of Pdx1 and Ngn3. In some cases, pancreatic endocrine progenitor cells and/or primitive beta-islet cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into ES cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into ES cells under the control of an inducible promoter and overexpression is induced by activation of the inducible promoter. In some aspects of the invention, pdx1 and ngn3 are integrated into ES cells under the control of an inducible promoter and mafA is transiently overexpressed. In some aspects of the invention, the ES cells further comprise a reporter molecule operably linked to a promoter active in pancreatic endocrine progenitor cells, primitive beta-islet cells or derivatives thereof but not expressed in primitive endoderm. In some cases, the reporter molecule is BLA and the pancreatic endocrine-specific promoter an ins1 promoter. In some aspects of the invention, the progression of ES cells to pancreatic endocrine progenitor cells can be monitored by expression of a reporter molecule operably linked to a promoter active in pancreatic endocrine progenitor cells or derivatives thereof but not expressed in primitive endoderm. In some aspects, the invention provides methods of producing pancreatic endocrine progenitor cells from pluripotent stem cells in monolayer. ES cells are induced to form definitive endoderm. In some cases, ES cells are induced to form definitive endoderm by incubating ES cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into ES cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into ES cells under the control of an inducible promoter and overexpression is induced by activation of the inducible promoter. In some aspects of the invention, pdx1 and ngn3 are integrated into ES cells under the control of an inducible promoter and mafA is transiently overexpressed. In some aspects of the invention, the ES cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter is an ins1 promoter. In some aspects of the invention, the progression of ES cells to pancreatic endocrine progenitor cells can be monitored by expression of a reporter molecule operably linked to a promoter active in pancreatic endocrine progenitor cells or derivatives thereof but not expressed in primitive endoderm. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm. The invention provides methods of producing pancreatic endocrine progenitor cells from iPS cells. In some aspects, iPS cells are first allowed to begin differentiation. Cells are then induced to form definitive endoderm. In some cases, cells are induced to form definitive endoderm by incubating cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into iPS cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into iPS cells under the control of an inducible promoter and overexpression is induced by activation of the inducible promoter. In some aspects of the invention, pdx1 and ngn3 are integrated into iPS cells under the control of an inducible promoter and mafA is transiently overexpressed. In some aspects of the invention, the iPS cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter an ins1 promoter. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm. The invention provides methods of producing pancreatic endocrine progenitor cells from iPS cells. In some aspects, iPS cells are first induced to form EBs. EBs are then induced to form definitive endoderm. In some cases, EBs are induced to form definitive endoderm by incubating EB cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into iPS cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into iPS cells under the control of an inducible promoter and overexpression is induced by activation of the inducible promoter. In some aspects of the invention, pdx1 and ngn3 are integrated into iPS cells under the control of an inducible promoter and mafA is transiently overexpressed. In some aspects of the invention, the iPS cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter an ins1 promoter. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm. In some aspects, the invention provides methods of producing pancreatic endocrine progenitor cells from iPS cells in monolayer. iPS cells are induced to form definitive endoderm. In some cases, iPS cells are induced to form definitive endoderm by incubating iPS cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into iPS cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into iPS cells under the control of an inducible promoter and overexpression is induced by activation of the inducible promoter. In some aspects of the invention, pdx1 and ngn3 are integrated into iPS cells under the control of an inducible promoter and mafA is transiently overexpressed. In some aspects of the invention, the iPS cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter is an ins1 promoter. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm. The present invention provides methods of screening compounds for their ability to modulate pancreatic endocrine cell function. Test compounds are contacted with pancreatic endocrine progenitor cells prepared from iPS cells by overexpressing Pdx1 and Ngn3 and determining any phenotypic or metabolic changes in the cell that result from being combined with the compound, and correlating the change with an ability of the compound to modulate secretion of pancreatic endocrine hormones; for example, but not limited to, insulin, glucagon, gherlin, or somatostatin. In some cases, pancreatic endocrine progenitor cells and/or primitive beta-islet cells produced from ES cells or iPS cells by overexpression of Pdx1, Ngn3 and MafA are used to screen compounds for their ability to modulate pancreatic endocrine function. In some aspects, the present invention provides methods of screening genes for their ability to modulate pancreatic endocrine cell function. Candidate genes may be identified by microarray analysis of pancreatic endocrine progenitor cells prepared from iPS cells by overexpressing Pdx1 and Ngn3. The genes of interest are introduced into pancreatic endocrine progenitor cells prepared from ES cells or iPS cells by overexpressing Pdx1 and Ngn3 and determining any phenotypic or metabolic changes in the cell that result from overexpression of the candidate gene. Phenotypic or metabolic changes may be correlated the change with an ability of the cell to secrete pancreatic endocrine hormones; for example, but not limited to, insulin, glucagon, gherlin, or somatostatin. In some aspects, the invention provides methods of screening compounds for their ability to modulate pancreatic endocrine cell function using a reporter cell system. Test compounds are contacted with pancreatic endocrine progenitor cells prepared from iPS cells by overexpressing Pdx1 and Ngn3, and comprising a reporter molecule operably linked to a promoter active in pancreatic endocrine progenitor cells or derivatives thereof but not expressed in primitive endoderm. The ability of test compounds to modulate pancreatic endocrine cell function is assessed by determining changes in expression of the reporter molecule. In some cases, pancreatic endocrine progenitor cells and/or primitive beta-islet cells produced from iPS cells by overexpression of Pdx1, Ngn3 and MafA are used to screen compounds for their ability to modulate pancreatic endocrine function. The invention provides methods of pancreatic cell therapy comprising administering to a subject in need of such treatment a composition comprising pancreatic endocrine progenitor cells prepared from iPS cells by overexpressing Pdx1 and Ngn3.

In some embodiments mesenchymal stem cells are used to generate exosomes and/or the IPS cells. More specifically, the culture of mesenchymal stem cells is subjected to centrifugation at 200-400×g for 5-20 minutes to remove remaining cells and cell debris; the supernatant is collected and subjected to high-speed centrifugation at 9,000-12,000×g for 60-80 minutes; and then the supernatant is again collected and subjected to ultracentrifugation at 90,000-120,000×g for 80-100 minutes to remove a supernatant, and thus exosomes remaining in the bottom layer can be obtained. According to a specific embodiment of the present invention, the culture of mesenchymal stem cells is collected, centrifuged at 300×g for 10 minutes to remove remaining cells and cell debris, and then the supernatant is collected, filtered using a 0.22-pm filter, and then centrifuged at 10,000×g and 4° C. for 70 minutes using a high-speed centrifuge. The centrifuged supernatant is again collected, and centrifuged at 100,000×g and 4° C. for 90 minutes using an ultracentrifuge to remove a supernatant, and thus exosomes remaining in the bottom layer were isolated.

In some embodiments of the invention, exosomes are utilized to provide an environment for dedifferentiation in the pancreatic microenvironment. In other embodiments exosomes are utilized to increase beta cell mass. Expansion of beta cell numbers by exosome administration is also envisioned within the current invention. In one embodiment exosomes are extracted from mesenchymal stem cells. Further embodiments encompass methods wherein mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Further embodiments encompass methods wherein mesenchymal stem cells do not express substantial levels of HLA-DR, CD117, and CD45. Further embodiments encompass methods wherein mesenchymal stem cells are derived from a group selected from: bone marrow, adipose tissue, umbilical cord blood, placental tissue, peripheral blood mononuclear cells, differentiated pluripotent stem cells, and differentiated progenitor cells. Further embodiments encompass methods wherein germinal stem cells express markers selected from a group comprising of: Oct4, Nanog, Dppa5 Rbm, cyclin A2, Tex18, Stra8, Dazl, beta1- and alpha6-integrins, Vasa, Fragilis, Nobox, c-Kit, Sca-1 and Rex1. Further embodiments encompass methods wherein adipose tissue derived stem cells express markers are selected from a group consisting of: CD13, CD29, CD44, CD63, CD73, CD90, CD166, Aldehyde dehydrogenase (ALDH), and ABCG2. Further embodiments encompass methods wherein adipose tissue derived stem cells are a population of purified mononuclear cells extracted from adipose tissue capable of proliferating in culture for more than 1 month. Further embodiments encompass methods wherein exfoliated teeth derived stem cells express markers selected from the group consisting of: STRO-1, CD146 (MUC18), alkaline phosphatase, MEPE, and bFGF.

1. A method of generating a population of insulin producing cells, said method comprising of: a) obtaining a somatic cell; b) dedifferentiating said somatic cell into a pluripotent stem cell; b) differentiating said pluripotent stem cell into a mesendoderm cell; c) differentiating said mesendoderm cell into an endoderm cell; and d) differentiating said endoderm cell into a beta cell, or beta-like cell.

2. The method of claim 1, wherein said pluripotent stem cells express one or more genes selected from the group consisting of: a) SSEA4; b) OCT4; c) NANOG; and d) PIM-1.

3. The method of claim 1, wherein said endoderm cell expresses PDX-1.

4. The method of claim 1, wherein said endoderm cell expresses Nkx6.1.

5. The method of claim 1, wherein said differentiation of said pluripotent stem cell into said insulin producing cell is performed in vivo.

6. The method of claim 5, wherein said dedifferentiation factor is OCT4 and k-RAS in the presence of mesenchymal stem cells.

7. The method of claim 6, wherein said mesenchymal stem cells are derived from a source selected from the group consisting of: a) bone marrow; b) umbilical cord blood; c) peripheral cord blood; d) menstrual blood; e) Wharton's Jelly; f) Perinatal Tissue.

8. The method of claim 7, wherein said mesenchymal stem cells are selected based on expression of CD73.

9. The method of claim 8, wherein said CD73 cells are expanded in the presence of platelet lysate.

10. The method of claim 1, wherein said insulin producing cells are generated in a manner so as to reduce immunogenicity.

11. The method of claim 1, wherein said insulin producing cells are generated in a manner so as to enhance tolerogenicity.

12. The method of claim 10, wherein said insulin producing cells are gene edited to remove immunogenic molecules.

13. The method of claim 11, wherein said immunogenic molecule is CD40.

14. The method of claim 11, wherein said tolerogenicity is enhanced by transfection with FoxP3.

15. The method of claim 11, wherein said tolerogenicity is enhanced by transfection with IL-10.

16. The method of claim 11, wherein said tolerogenicity is enhanced by transfection with HLA-G.

17. The method of claim 1, wherein said pluripotent stem cells are created using non-integrating viral vectors.

18. The method of claim 17, wherein said pluripotent stem cells result in no karyotype abnormality.

19. The method of claim 18, wherein said pluripotent stem cells can be differentiated and demonstrate no adventitial viral components.

20. The method of claim 19, wherein said pluripotent stem cells can be differentiated and produce functional insulin.
